(19) United States
(12) Patent Application Publication
(10) Pub. No.: US 2009/0082822 A1
(43) Pub. Date: Mar. 26, 2009

(54) Transpedicular, Extrapedicular and Transcorporeal Approaches to the Intervertebral Discs

(76) Inventor: Said G. Osman, Russellville, AL (US)

Correspondence Address: NIXON & VANDERHYE, PC, 901 NORTH GLEBE ROAD, 11TH FLOOR, ARLINGTON, VA, 22203, US

(21) Appl. No.: 12/248,264

(22) Filed: Oct. 9, 2008

Related U.S. Application Data
(63) Continuation-in-part of application No. 12/234,891, filed on Sep. 22, 2008.
(60) Provisional application No. 60/978,547, filed on Oct. 9, 2007.
(60) Provisional application No. 60/973,899, filed on Sep. 20, 2007.

(51) Int. Cl. A61F 5/00; A61B 19/00; A61M 29/00; A61F 2/44

(52) U.S. Cl. 606/86 R; 128/898; 606/192; 623/17.16

(57) ABSTRACT

A method of surgically accessing intervertebral disc space involves a less invasive technique than prior procedures. The method avoids violation of the disc annulus and utilizes the annulus for containment of artificial disc material. The less invasive technique results in less blood loss and minimization of surgical trauma. The method includes the steps of identifying a target disc space, preparing portals adjacent the disc space, introducing a guide wire into each of the portals, using the guide wires, removing a core of bone with a trephine along each of the portals, and accessing the intervertebral disc space for performing intradiscal procedures.

### CROSS-REFERENCES TO RELATED APPLICATIONS

This application claims the benefit of U.S. Provisional Patent Application Ser. No. 60/978,547, filed Oct. 9, 2007, the entire content of which is herein incorporated by reference.

This application is also a continuation-in-part (CIP) of U.S. patent application Ser. No. 12/234,891, filed Sep. 22, 2008, pending, which claims the benefit of U.S. Provisional Patent Application Ser. No. 60/973,899, filed Sep. 20, 2007, the entire contents of each of which are hereby incorporated by reference in this application.

### STATEMENT REGARDING FEDERALLY SPONSORED RESEARCH OR DEVELOPMENT

(NOT APPLICABLE)

### BACKGROUND OF THE INVENTION

The invention relates to disc arthroplasty (or partial disc replacement) and, more particularly, to a nucleus replacement that mimics a native annulus in shape and function for use in partial disc arthroplasty.

The gold standard for the lumbar and cervical disc degeneration is currently fusion of the diseased motion-segment. The outcomes of the various fusion techniques have generally been satisfactory. Elimination of motion at a joint naturally produces stiffness and this in turn leads to transfer of forces normally absorbed by the motion-segments to the adjacent joints. As a result of the increased forces to which the adjoining joints are subjected, accelerated wear and tear takes place, setting the scene for arthritis at those joints. These observations have lead, therefore, to a search for alternative surgical treatments that would alleviate pain and restore function while preserving motion. Recently, a number of artificial disc prostheses have come into clinical use both in the lumbar spine and the cervicle spine. The results of these procedures, on medium term follow-ups, have been equivalent to fusion. Most of the devices for total disc replacement are performed anteriorly (through the belly) for the lumbar spine and the cervicle spine. The surgical trauma for the lumbar disc replacement is significant, and the approach has been associated with serious complications.

The current minimally invasive partial disc replacement devices are at experimental stages in the USA, and most have significant design flaws, notably instability of the devices in the disc spaces, and reliance on the already compromised annulus fibrosus to contain the devices. Furthermore, implantation of the current devices requires further violation of the annulus to implant the devices.

Approaches to the disc spaces have evolved over the last century and continue multiplying with technological advances. Currently, open approaches to the lumbar spine include (a) anterior—both transperitoneal and retroperitoneal, (b) posterior—access through the spinal canal and trans-foraminal routes, and (c) more recently, direct lateral approach for interbody fusion and implantation of artificial discs. While the access routes and the level of surgical trauma and risks to neural injury may differ with these various approaches, they all have one thing in common; that is, all of them require violation of the disc annulus to access the disc space.

While the violation of the disc annulus may not matter much for the fusion procedures, when performing disc replacement procedures, it is desirable to retain the annulus since the stability of the artificial disc, to some extent, depends on the integrity of the native annulus. This is particularly true for the partial disc replacement devices that do not have adequate fixation within the disc space.

### BRIEF SUMMARY OF THE INVENTION

The degenerate disc that is intended for replacement already has its structure compromised by disease even before surgical intervention. The described embodiments avoid violation of this already compromised but vital structure, by accessing the disc space through the adjacent bone structures. These access portals include the pedicle and the vertebral body that may be accessed posterolaterally, laterally or anteriorly. Both minimally invasive and open techniques may be utilized.

In an exemplary embodiment, a method of surgically accessing intervertebral disc space in a patient includes the steps of (a) identifying a target vertebra and its pedicles; (b) preparing a posterolateral portal and a contralateral portal; (c) introducing a guide wire into each of the portals; (d) using the guide wires, removing a core of bone with a trephine from at least the target vertebra and an end plate via each of the portals; and (e) accessing the intervertebral disc space for performing intradiscal procedures. For a transpedicular approach, step (d) may be practiced by removing the core of bone from the pedicle.

Step (b) is preferably practiced by introducing a trocar and a cannula into the pedicle in alignment with the patient cephalad disc space. In this context, step (c) may be practiced after removing the trocar and by introducing the guide wire through the cannula. The method may further include, after step (c), removing the cannula, introducing a cannulated series of tissue dilators over the guide wire until the dilators contact the pedicle, and removing inner ones of the tissue dilators.

In one embodiment, the method may further include, after step (e), plugging holes resulting from step (d) using one of the core of bone or an artificial plug.

In another exemplary embodiment, a method of surgically accessing intervertebral disc space in a patient includes the steps of (a) identifying and marking a level of a target disc, disc endplates, and vertebral body anterior and posterior cortices; (b) preparing a cephalad portal and a caudal portal on the level identified and marked in step (a); (c) performing an anterior sweep to insert an obturator through the caudal portal until the obturator contacts the vertebral body at a desired location; (d) introducing a guide wire through the obturator into the vertebral body and exiting the endplates on an antero-posterior projection; (e) using the guide wires, removing a core of bone with a trephine from either side of the vertebral body and the endplates; and (f) accessing the intervertebral disc space for performing intradiscal procedures.

The method may further include, prior to step (a), placing the patient in a lateral decubitus position on a radioluscent operating table with a wedge under the patient lower flank to open up an upper lumbar space.

Preferably, steps (a) and/or (b) are practiced under fluoroscopic visualization. Step (c) may be practiced by performing the anterior sweep to insert the obturator at a junction of lower and middle thirds of the vertebral body. The method may also include, after step (d), inserting a cannula over the obturator as a tissue protector, and removing the obturator. Still further, the method may additionally include, after step (e), docking a cannula into the vertebral body up to the end plates to act as access channels for performing the intradiscal procedures.

In yet another exemplary embodiment, the method includes the steps of (a) identifying a target disc space; (b) preparing portals adjacent the disc space; (c) introducing a guide wire into each of the portals; (d) using the guide wires, removing a core of bone with a trephine along each of the portals; and (e) accessing the intervertebral disc space for performing intradiscal procedures.

### BRIEF DESCRIPTION OF THE DRAWINGS

These and other aspects and advantages will be described in detail with reference to the accompanying drawings, in which:

FIGS. 1A-1C show exemplary shapes of the nucleus replacement;

FIGS. 2A-2C illustrate configurations of the replacement jacket;

FIGS. 3A and 3B show exemplary load-bearing designs for insertion into the jacket;

FIGS. 4A-4E show alternative configurations for anchoring the jacket in the disc space;

FIGS. 5A and 5B demonstrate implantation of the load-bearing device in the jacket; and

FIGS. 6A-6H and FIG. 7 illustrate approaches to surgically accessing intervertebral disc space.

### DETAILED DESCRIPTION OF THE INVENTION

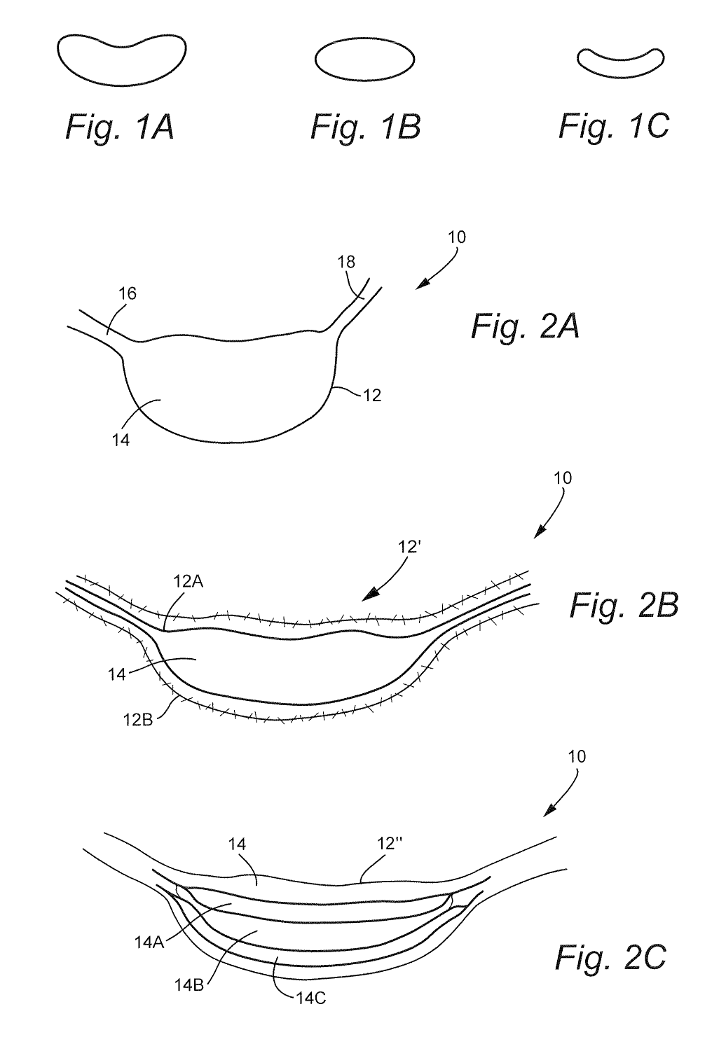

With reference to FIGS. 1A-1C and FIGS. 2A-2C, a nucleus replacement 10 mimics a native annulus in shape and function for use in partial disc arthroplasty. As shown in FIGS. 1A-1C, the replacement 10 can take numerous shapes including, without limitation, bean shaped, oval shaped, cylindrical shaped (or more accurately banana-shaped), and the like. The replacement 10 generally includes a jacket 12 that includes a compartment 14 and first and second anchoring limbs 16, 18. As described in more detail below, the anchoring limbs 16, 18 facilitate positioning of the jacket 12 in the disc space and enable the jacket 12 to be secured in the disc space. In FIG. 2A, the jacket 12 includes a single compartment 14. The jacket 12 may be formed of numerous suitable materials, including, without limitation, elastic or inelastic fabric, which may be pervious or impervious. FIG. 2B shows an embodiment utilizing a double jacket 12′ including an inner jacket 12A and an outer jacket 12B. The outer jacket 12B serves as a restraint for the nucleus replacement 10, and the inner jacket 12A includes the compartment 14. The outer jacket 12B may be formed of or impregnated with a material that serves to encourage bone ingrowth. The outer jacket 12B is preferably formed of a synthetic, biologically active and/or inert fabric. The inner jacket 12A is preferably formed of an impervious synthetic material.

FIG. 2C shows yet another alternative for the jacket 12″, which may be a single jacket as in FIG. 2A or a double jacket as in FIG. 2B. The compartment 14 is divided into a plurality of sub-compartments, e.g., 14A, 14B, 14C, which are preferably interconnected. The multi-compartment construction enables the replacement 10 to better fit the geometry of the spine. The multiple compartments 14A-14C are designed to allow distribution of forces across the disc in a uniform manner while maintaining the lordosis of the disc. The lordosis is achieved by making the anterior compartment larger than the posterior compartment.

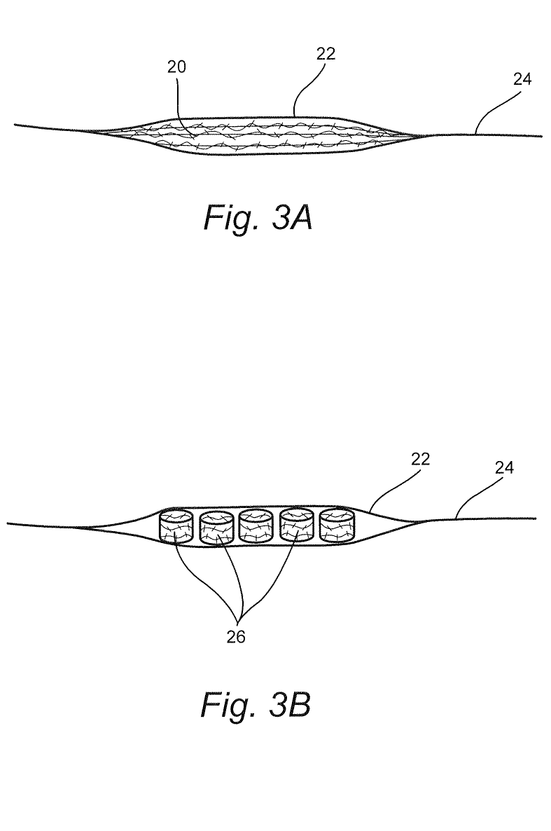

A shock-absorbing material/insert is injectable into the compartment 14 after installing the jacket 12 into the disc space. In the multi-compartment embodiment illustrated in FIG. 2C, with the compartments 14A-14C interconnected, the shock-absorbing material is preferably flowable between the compartments. Many materials are suitable for the shock-absorbing material, and it is preferred that the shock-absorbing material includes characteristics that absorb loads on the replacement 10. For example, the material may be a liquid, a gelatinous osmotically active material and/or a biological material. Alternatively, with reference to FIGS. 3A and 3B, the shock-absorbing material may comprise bundles of fabric or fibers 20 contained within a sub-jacket 22 including leading and trailing tails 24 that are used to pull the bundle into the jacket compartment 14. Alternatively, the sub-jacket 22 may include cylinders of fabric 26 that provide the shock-absorbing function. As would be appreciated by those of ordinary skill in the art, other materials and/or shapes of fabric and the like may be suitable, and the invention is not meant to be limited to the illustrated exemplary embodiments.

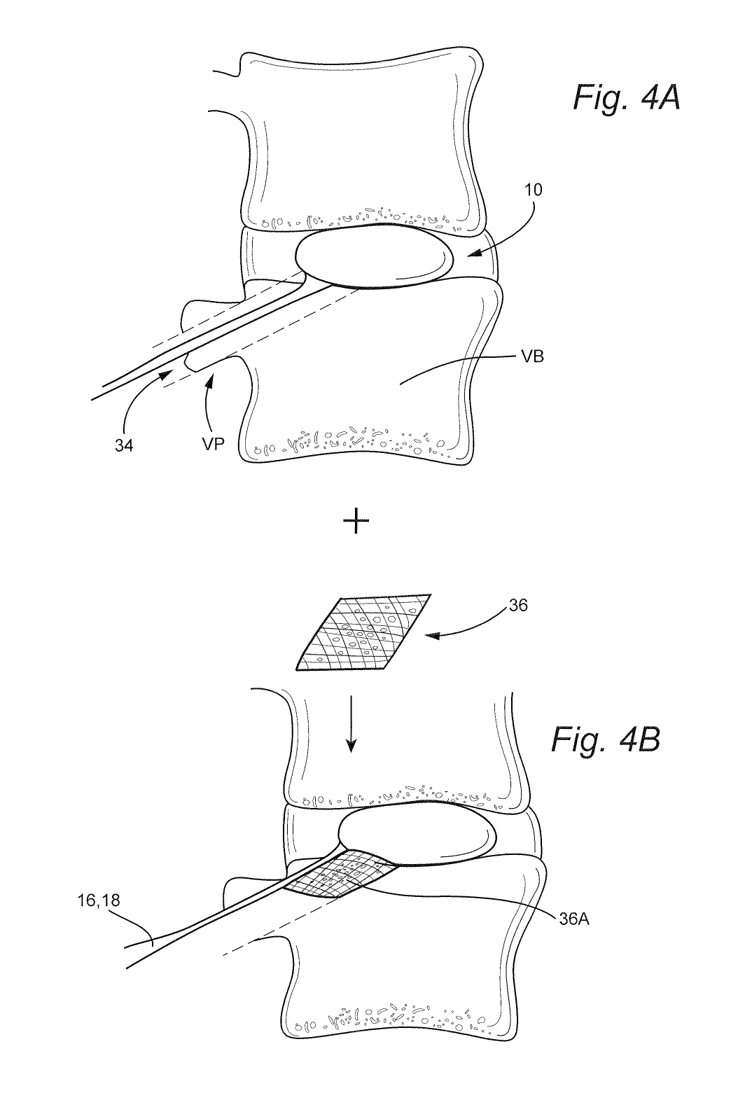

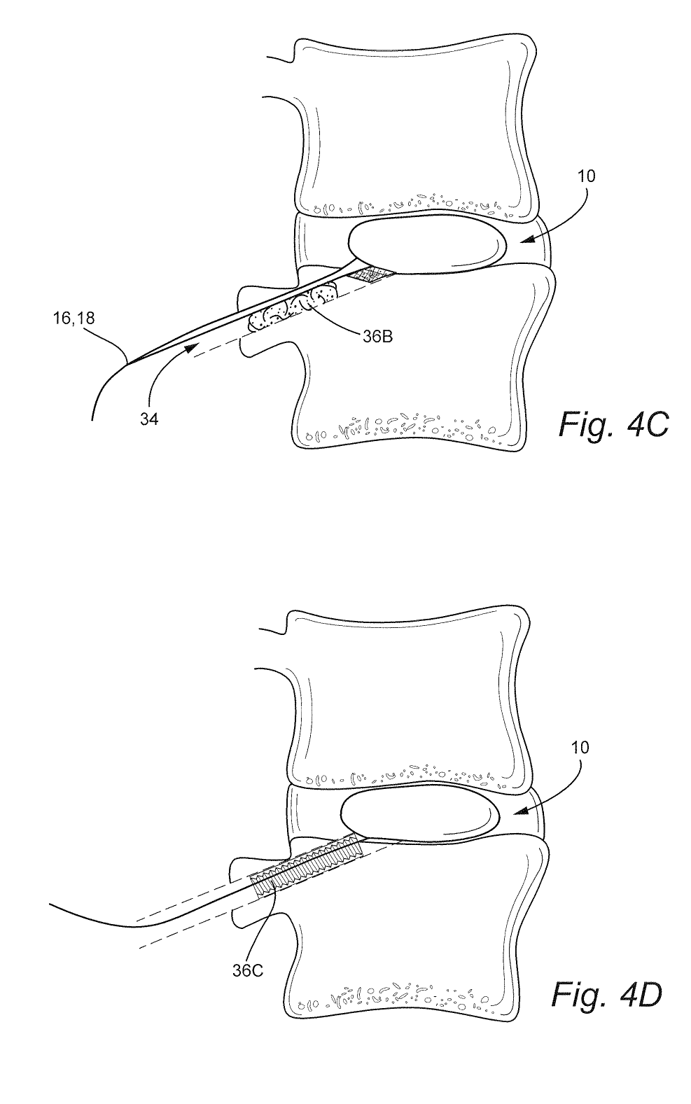

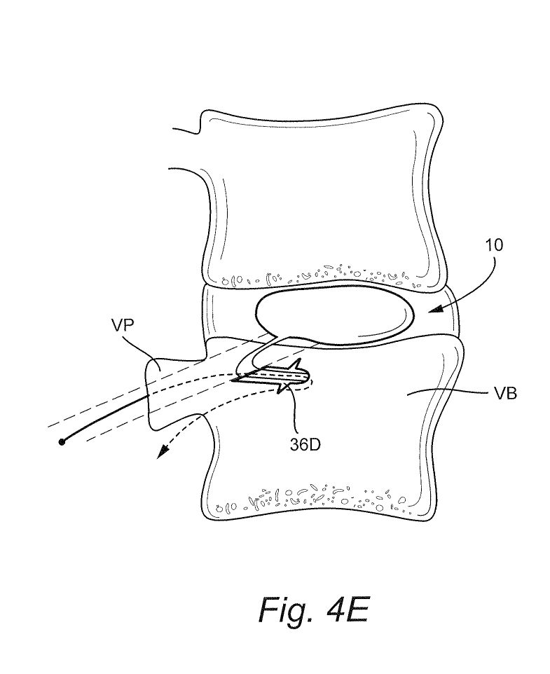

With reference to FIGS. 4A-4E, it is preferable to fix the replacement 10 in the disc space. Such fixation may be effected by fibrous ingrowth into the outer wall of the jacket 12 or bone ingrowth into the jacket 12. As noted, the jacket 12 or outer jacket 12B may be formed of a material that encourages bone or fibrous ingrowth. In a preferred construction, the replacement 10 is secured using intraosseous anchors that secure the anchoring limbs 16, 18 in an operating channel 34 through which the replacement 10 is inserted. In preparing the disc space, it is preferable to form an operating channel 34 through the vertebral pedicle VP and/or the vertebral body VB into the disc space (see FIG. 4A). After securing cannulas in the operating channel 34, the disc space can be cleaned using a disc reamer or the like through the cannulas. The replacement 10 is inserted through the operating channel 34, preferably under X-ray inspection. Alternatively or additionally, the first and second anchoring limbs 16, 18 may be provided with a radio-opaque marker to facilitate insertion of the replacement 10.

Once installed, the intraosseous anchors 36 can be inserted to secure the anchoring limbs 16, 18 via the operating channel 34. FIG. 4B shows a bone plug 36A disposed in the operating channel 34 to secure the anchoring limbs 16, 18. FIG. 4C illustrates the use of bone cement 36B inserted into the operating channel 34 for fixing the anchoring limbs 16, 18. FIG. 4D shows interferential screw fixation utilizing a screw 36C to secure the replacement 10 in the disc space. FIG. 4E shows the use of known suture anchors 36D in the vertebral body VB or vertebral pedicle VP.

With the anchoring structure as a screw 36C for interferential screw fixation, the screw 36C may be cannulated and used as a channel for access to the liquid filled replacement 10 for replenishment post-operatively at pre-defined intervals. The jacket 12 may be self-sealing in this design. Alternatively, the anchoring limbs 16, 18 may be fixed with a set-screw into the cannulated pedicle VP or intraosseous screw and used as a port for injectate.

The implantation approaches for the disc prosthesis or replacement 10 may be transpedicular, extrapedicular, transcorporeal, or transdiscal (for partial replacement of a surgical disc). The implantation technique is important for this minimally invasive device. Whether liquid, gel or fabric is used for the shock-absorbing material, the jacket 12 is inserted empty and subsequently armed with the shock-absorbing material in situ. As a consequence, a lesser amount of bone is removed in installing the replacement 10, thereby reducing the risk of fracture.

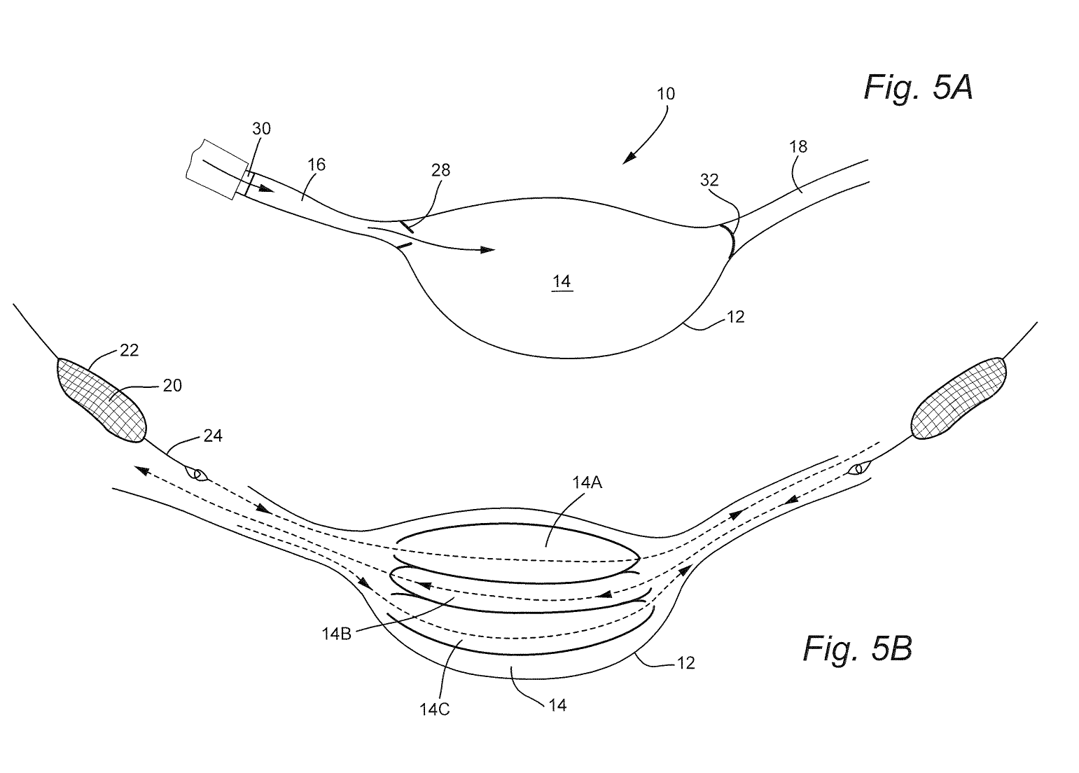

With reference to FIG. 5A, when liquids or osmotically active material is used as the shock-absorbing material, the replacement 10 may be additionally provided with a valve 28 at one end of the jacket 12 to allow inflow only of the fluid. In this arrangement, a nozzle 30 is provided at an end of the first anchoring limb 16 so that the material can be injected via a syringe. An opposite end of the jacket 12 adjacent the second anchoring limb 18 includes a seal 32 to prevent the liquid from escaping the compartment 14. The valve 28 also serves to prevent the extrusion of gelatinous material outside the nucleus compartment 14.

In using gel and fabrics as the shock-absorbing material, with reference to FIG. 5B, the shock-absorbing material can be housed in the mini- or sub-jackets 22 including the leading and trailing tails 24 at respective ends for pulling the device into the jacket 12. The side opposite the entrance is preferably sealed to prevent extrusion of the shock-absorbing material. In one design, openings in adjacent compartments 14A, 14B, 14C may be made to alternate between the right and left sides to facilitate insertion of the shock-absorbing material via the sub-jackets 22 in the multi-compartment embodiment.

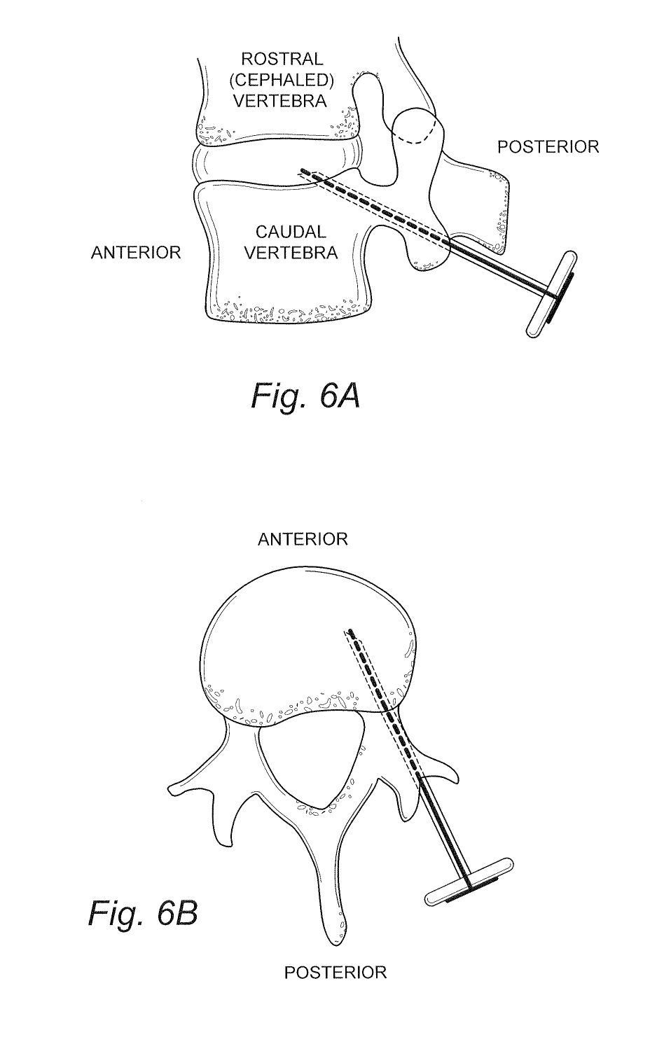

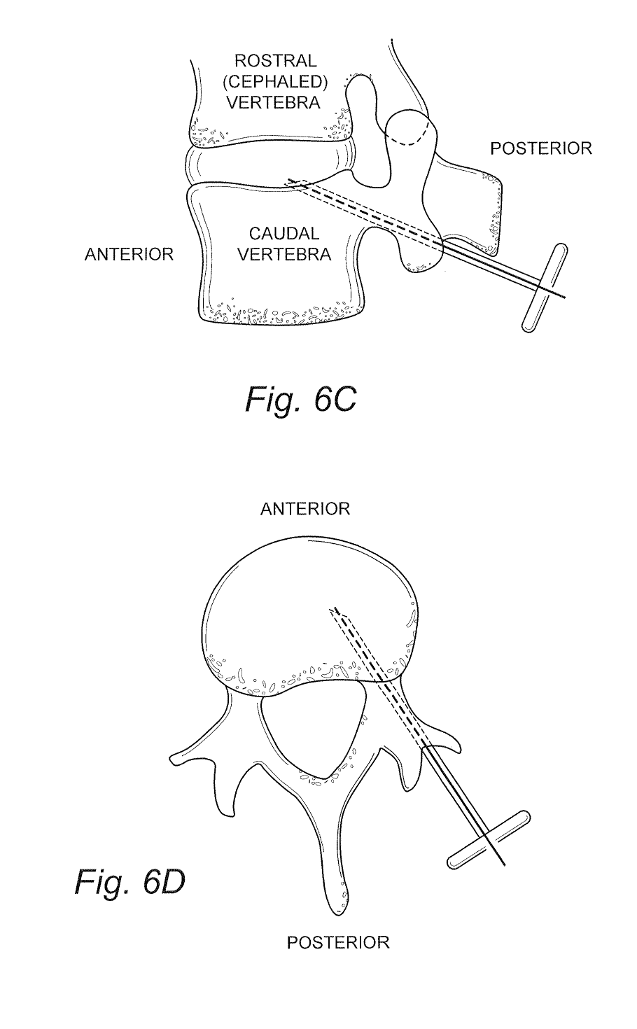

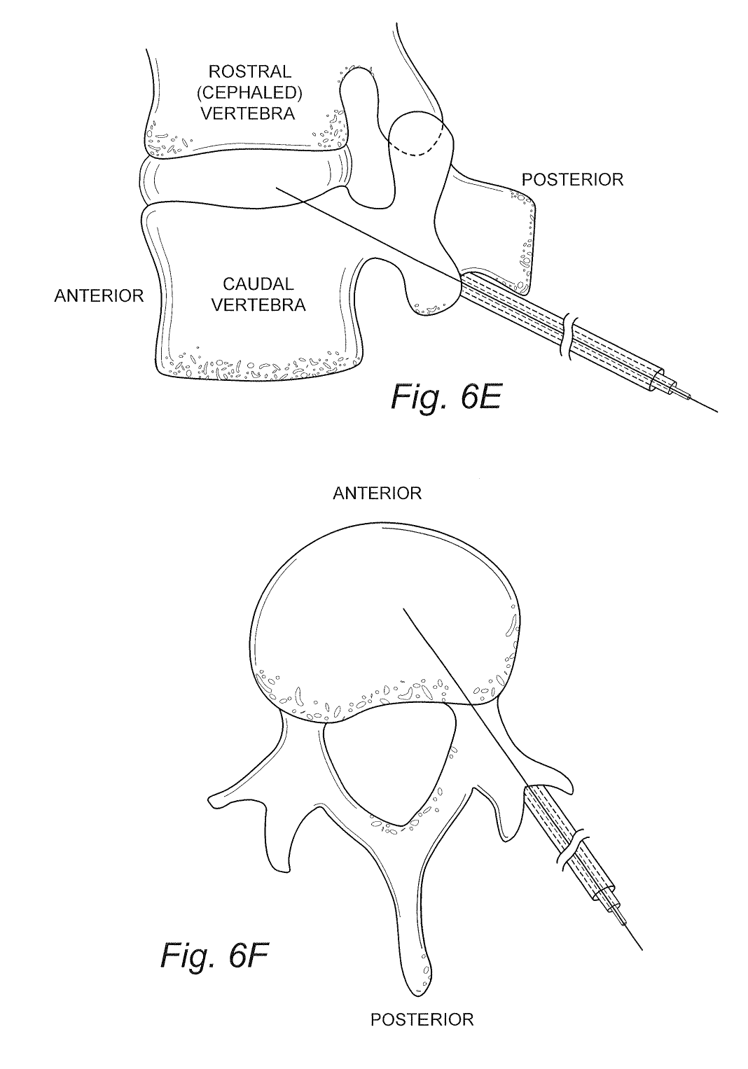

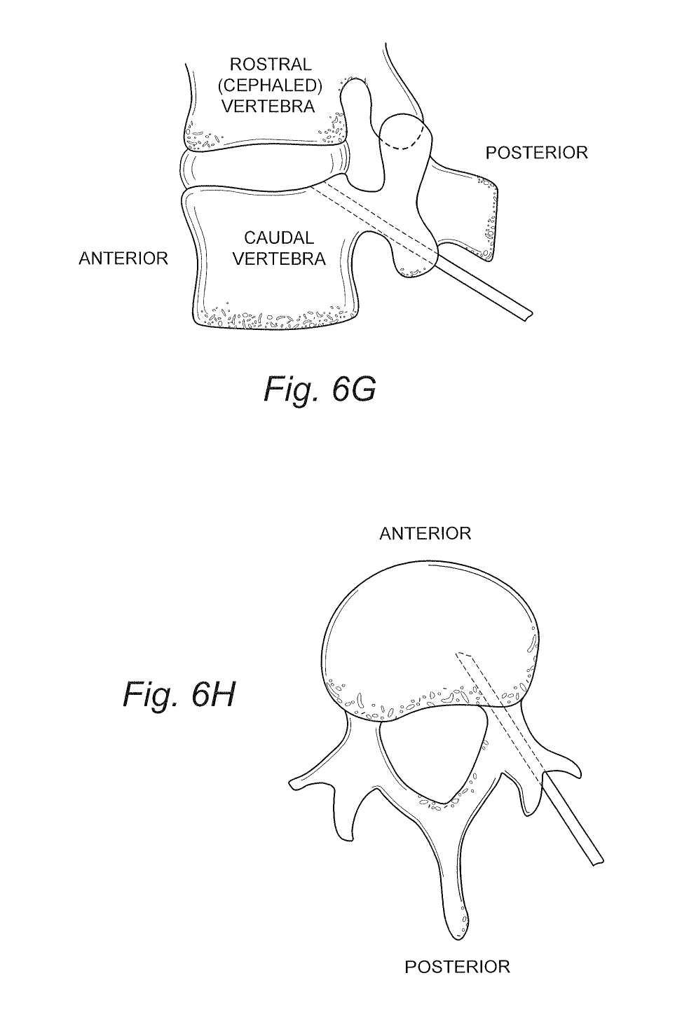

FIGS. 6A-6H illustrate approaches to surgically accessing intervertebral disc space. The indications for these approaches include partial disc replacement, interbody fusions and debridement of the disc spaces, especially when dealing with vertebral osteomyelitis and disc space infection.

Several approaches to accessing the disc space in a patient will be described, including a transpedicular approach, an extrapedicular approach, and a lateral transosseous approach. Each approach shares the steps of identifying a target disc space, preparing portals adjacent the disc space, and introducing a guide wire into each of the portals. Using the guide wires, a core of bone is removed with a trephine along each of the portals. With the core of bone removed, the intervertebral disc space is accessible for performing intradiscal procedures. A more detailed description of each approach is provided below.

In the transpedicular approach, (a) the patient is placed in a prone position on a radiolucent table; (b) routine skin preparation and sterile draping is performed as for posterior approach to the lumber or thoracic spine; (c) under fluoroscopic visualization, the target vertebra and its pedicles are identified and marked on the skin; (d) a posterolateral portal is developed at the appropriate site, and an assembly of sharp trocar and cannula are introduced into the pedicle aiming to enter the cephalad (or caudal) disc space at about the level of the middle ⅓ of the disc space as visualized on the lateral fluoroscopic view (the procedure is repeated on the contralateral side); (e) the trocar is removed and a guide wire is introduced through the cannula into the disc space, and the cannula is removed; (f) a cannulated series of tissue dilators are introduced over the guide wire until they contact the pedicle; (g) the inner dilators are removed leaving the outermost dilator in place; (h) a trephine is use to remove a core of bone of a given diameter from the pedicle, vertebral body and the end-plate; (i) an access cannula of appropriate size is placed in the channel created as above for intradiscal procedures; and (j) steps (e), (f), (h), and (i) are repeated on the contralateral side.

In step (h), the core of bone may be used at end of the procedure to plug the hole in the vertebral body and the end-plate. Alternatively, the core of bone may be discarded, and an artificial plug with ceramic, bone allograft, or metal screw used instead to give structural support at the defect in the end-plate.

In the extrapedicular approach, the steps in the procedure are identical to the transpedicular approach except that it is placed outside the pedicle instead of through the pedicle. The indications for this approach include, for example, hypoplastic pedicle, malformation of pedicle, and mal-orientation of the pedicle.

In the lateral transosseous approach, the approach is through the vertebral bodies adjacent the disc space. It is a direct lateral approach to the disc space and may be performed by open retroperitoneal approach or minimally invasive lateral approach. In the minimally invasive approach, (a) the patient is placed in a lateral decubitus position on a radiolucent operating table with a wedge under the lower flank to open up the upper lumbar space; (b) under fluoroscopic visualization, the level of the target disc is marked on the skin along with the endplates and anterior and posterior cortices of the vertebral bodies; (c) under fluoroscopic visualization, two portals are developed—one cephalad and one caudal to the disc and on the mid lateral disc line previously described; (d) using a bullet shaped cannulated obturator, an anterior sweep technique is used to insert the obturator to the junction of the lower and middle thirds of the vertebral body; (e) while holding the obturator at the desired position, a guide wire is inserted through it and into the vertebral body to exit the end-plate at the junction of the lateral and middle thirds on the antero-posterior projection fluoroscopically; (f) a cannula is inserted over the obturator as tissue protector; (g) the obturator is removed while holding the guide wire and the cannula in place; (h) a trephine is used to take a core of bone from either side along with the endplates; and (i) a cannula is docked into the vertebral bone up to the end plates to act as the access channels for a subsequent procedure.

In step (d), the anterior sweep technique involves the obturator through the portal toward the imaginary line joining the adjacent transverse process tips. At the imaginary intertransverse line, the obturator is swept anteriorly and medially until it contacts the vertebral body at the desired spot. Care is taken to avoid the waist of the vertebra where there are the segmental vessels.

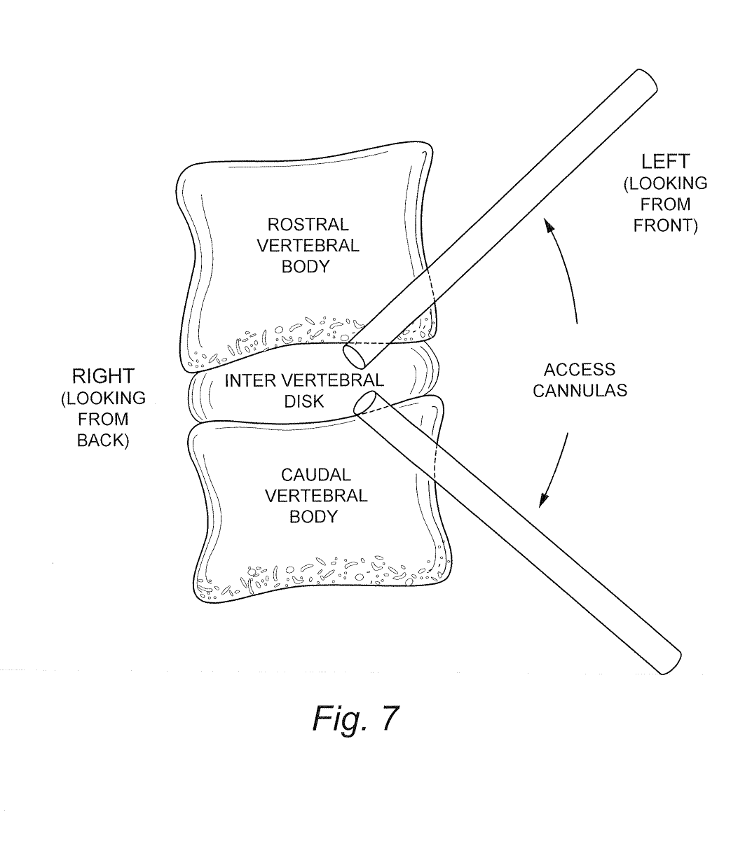

The transcorporeal/extrapedicular approach is illustrated in FIG. 7.

The method of the described embodiments for surgically accessing the invertebral disc space involves a less invasive technique than prior procedures. The method avoids violation of the disc annulus and utilizes the annulus for containment of artificial disc material. The less invasive technique results in less blood loss and minimization of surgical trauma.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of surgically accessing intervertebral disc space in a patient, the method comprising:
(a) identifying a target vertebra and its pedicles;
(b) preparing a posterolateral portal and a contralateral portal;
(c) introducing a guide wire into each of the portals;
(d) using the guide wires, removing a core of bone with a trephine from at least the target vertebra and an end plate via each of the portals; and
(e) accessing the intervertebral disc space for performing intradiscal procedures.

2. A method according to claim 1, wherein for a transpedicular approach, step (d) is practiced by removing the core of bone from the pedicle.

3. A method according to claim 1, wherein step (b) is practiced by introducing a trocar and a cannula into the pedicle in alignment with the patient cephalad disc space.

4. A method according to claim 3, wherein step (c) is practiced after removing the trocar and by introducing the guide wire through the cannula.

5. A method according to claim 3, further comprising, after step (c), removing the cannula, introducing a cannulated series of tissue dilators over the guide wire until the dilators contact the pedicle, and removing inner ones of the tissue dilators.

6. A method according to claim 1, further comprising, after step (e), plugging holes resulting from step (d) using one of the core of bone or an artificial plug.

7. A method of surgically accessing intervertebral disc space in a patient, the method comprising:
(a) identifying and marking a level of a target disc, disc endplates, and vertebral body anterior and posterior cortices;
(b) preparing a cephalad portal and a caudal portal on the level identified and marked in step (a);
(c) performing an anterior sweep to insert an obturator through the caudal portal until the obturator contacts the vertebral body at a desired location;
(d) introducing a guide wire through the obturator into the vertebral body and exiting the endplates on an antero-posterior projection;
(e) using the guide wires, removing a core of bone with a trephine from either side of the vertebral body and the endplates; and
(f) accessing the intervertebral disc space for performing intradiscal procedures.

8. A method according to claim 7, further comprising, prior to step (a), placing the patient in a lateral decubitus position on a radioluscent operating table with a wedge under the patient lower flank to open up an upper lumbar space.

9. A method according to claim 7, wherein step (a) is practiced under fluoroscopic visualization.

10. A method according to claim 7, wherein step (b) is practiced under fluoroscopic visualization.

11. A method according to claim 7, wherein step (c) is practiced by performing the anterior sweep to insert the obturator at a junction of lower and middle thirds of the vertebral body.

12. A method according to claim 7, further comprising, after step (d), inserting a cannula over the obturator as a tissue protector, and removing the obturator.

13. A method according to claim 7, further comprising, after step (e), docking a cannula into the vertebral body up to the end plates to act as access channels for performing the intradiscal procedures.

14. A method of surgically accessing intervertebral disc space in a patient, the method comprising:
(a) identifying a target disc space;
(b) preparing portals adjacent the disc space;
(c) introducing a guide wire into each of the portals;
(d) using the guide wires, removing a core of bone with a trephine along each of the portals; and
(e) accessing the intervertebral disc space for performing intradiscal procedures.
